(19) United States
(12) Patent Application Publication
(10) Pub. No.: US 2010/0129436 A1
(43) Pub. Date: May 27, 2010

(54) SUBTANCES FOR THE PROTECTION OF CELLS AND/OR TISSUES

(76) Inventors: Ralf Losel, Schwabach (DE); Benito Yard, Dackenheim (DE); Grietje Beck, Heidelberg (DE)

Correspondence Address: Ballard Spahr LLP, SUITE 1000, 999 PEACHTREE STREET, ATLANTA, GA, 30309-3915, US

(21) Appl. No.: 12/452,889

(22) PCT Filed: Jul. 10, 2008
(86) PCT No.: PCT/EP2008/005651

§ 371 Date: Jan. 27, 2010

(30) Foreign Application Priority Data
Jul. 30, 2007 (DE) 102007035642.2

(51) Int. Cl. A01N 1/02; A61K 31/137; A61K 31/16; A61K 9/127

(52) U.S. Cl. 424/450; 435/1.1; 514/649; 514/613; 514/529

(57) ABSTRACT

The invention relates to substances that are suitable for protecting cells and/or tissues.

### FIELD OF THE INVENTION

The invention relates to substances which are suitable for the protection of cells and/or tissue.

### BACKGROUND OF THE INVENTION

Organs and tissues of mammals or other eukaryotic cells can be exposed to a wide variety of damaging influences. Precisely the cells of higher organisms are particularly susceptible, with damage frequently occurring in particular when cells or tissues are removed from the organism, such as for example in cell cultures or during transplants. Moreover, damage also occurs when the original, environment of the cells or organs is modified, e.g. by surgical intervention or pathological processes.

Particularly severe damage to cells or tissues in a mammal occurs under ischaemic conditions. The term ischaemia refers to the pathologically restricted or blocked flow of blood through a tissue as a result of an inadequate arterial blood supply, which leads to an under-supply of oxygen to the cells or tissue. Despite the reduced oxygen supply, paradoxical oxidative damage to the cells or tissue is often found in this case. Damage to cells or tissues caused by ischaemia can often occur during surgical procedures and is responsible for high complication rates. It is an object of the invention to counteract this risk.

Of particular importance is the protection of cells and tissues in the field of transplant surgery. From the removal of an organ to its insertion into the recipient, it is important to protect its functions as far as possible.

Since cells or tissues can only be transplanted immediately in situ in the rarest cases, it will always be necessary to preserve the cells or the tissue. Often in this case the tissue is stored at low temperatures after removal, resulting in a reduction in metabolism. However, this can lead to severe damage to the tissue through the action of the cold itself. This is particularly true of internal organs which are stored at low temperatures. One example is the kidneys, where the cold damages the endothelial cells of the kidneys, leading to the loss of the barrier function, which is associated with a markedly increased risk of immunological complications or functional lesions. While it is true that the active substances used up to now in the context of studies or experimentally for the prevention of cryopathy, such as dopamine or dobutamine, display a protective effect, however, a very high concentration is needed for this purpose. When used in animals or humans, therefore, a very marked haemodynamic effect is observed after just a short period, which generally leads to complications and is therefore undesirable. When dopamine or dobutamine acts on cell cultures, the metabolism is significantly altered in such a way that the cells no longer exhibit their proper functionality and are therefore unsuitable for transplant.

Another way of preserving cells or tissues, in particular during transplant, is the perfusion of the tissue or cells with solutions containing preservatives. Thus, solutions to increase the life of transplant tissues are described which contain PHB and PHB-folic acid antagonists in combination. Furthermore, DE 295 04 589 U1 describes the use of benzoic acid and its derivatives for this purpose, optionally in combination with other active substances. Elsewhere, reference is made to the use of adrenalin or carvedilol.

An optimum substance which on the one hand protects the cells or the tissue adequately from ischaemic damage and on the other hand achieves the desired protective effect in a low concentration so that no haemodynamic effects occur, the substance being neither harmful to health nor damaging to the environment, has not been found up to now.

The object of the invention was therefore to find a substance which protects cells and tissues in vivo, but particularly during storage and transport, i.e. ex-vivo, and in particular preserves tissues to be transplanted or removed cells from ischaemic damage or cryopathy, or reduces these. Furthermore, it must be possible to use the substance in a low concentration and no haemodynamic or other undesirable activity must occur.

These objects are achieved by a substance with the features according to claim 1. The subclaims contain advantageous developments.

### DETAILED DESCRIPTION OF THE INVENTION

Surprisingly, it has now been found that an aromatic system containing at least one aromatic ring which has two substituents R₁ and R₂ having a reducing effect and a further substituent R₃, such that the log P of the molecule is at least 2.5, can protect cells or tissues. The substance according to the invention is illustrated in the following general formula (I):

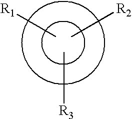

wherein the double circle represents an aromatic system with 6 to 18 C atoms, which carries at least the substituents R₁, R₂ and R₃, wherein R₁ and R₂ are each selected from the group consisting of OH, SH and NH₂, which may also be present in protected form, and R₃ is a hydrophobic group, wherein log P of the substance is at least 2.5.

The aromatic system can be built up both from aromatic rings which bear exclusively carbons as ring atoms and from those which also have hetero atoms, provided that they are biologically compatible. Suitable examples are aromatic rings in the aromatic system containing carbazoles and derivatives thereof as hetero atoms; aromatics containing only carbon are preferred.

The aromatic system has one or more aromatic rings which can be condensed together. Preferred examples are phenyl, naphthalene and anthracene. The aromatic system can carry additional substituents in addition to the substituents R₁, R₂ and R₃, which are inert in relation to the desired properties and may optionally stabilise or activate the system. Preferably, apart from R₁, R₂ and R₃ no other substituents are bonded.

The aromatic system which carries at least the substituents R₁, R₂ and R₃ is preferably distinguished by the fact that it contains 5-, 6- or 7-membered rings. Rings with a size of 5 to 7 atoms have a high ring stability, and so they exhibit reduced internal stresses even with a high degree of substitution of the aromatic ring. In addition, these aromatic systems are readily obtainable, well tested and thus safe, in the sense of not harmful to health or damaging to the environment.

Also preferred is an aromatic system having 1 to 3 rings. In principle, it is true that compounds can also be used, the aromatic system of which contains more rings, but it has been shown that in particular smaller aromatic systems with only 1 to 3 rings can penetrate through the cell wall more readily owing to their small size.

The substituents R₁ and R₂ are selected from the group consisting of OH, SH and NH₂, optionally in protected form, it being possible for any combination of these residues to be present. Preferably, R₁ and R₂ are each OH. The groups can be protected with a protective group in order to protect them from harmful reactions during storage.

The residues R₁ and R₂ are each bonded to an aromatic ring in the aromatic system, either in ortho or para position to one another. It is presumed that the reducing action of the two functional groups, and thus the protection of the tissue from oxidative damage, is reinforced precisely by this selective position of the two substituents R₁ and R₂ to one another. It may be assumed that, owing to the conformation of the aromatic ring in ortho or para position, the two functional groups, i.e. the substituents R₁ and R₂, point in the same direction and thus their function is synergistically reinforced. The aromatic system is particularly preferably derived from 2,5-dihydroxybenzoic acid, 3,4-dihydroxybenzoic acid, 2,5-dihydroxyphenethylamine or 3,4-dihydroxyphenethylamine.

However, the substance according to the invention can only protect cells or tissues from damaging influences if the substance is formed in such a way that the log P of the substance is at least 2.5. Log P is an empirically calculated parameter and can be calculated mathematically from the structure of a substance, where P represents the coefficient of distribution of the substance in question between n-octanol and water, i.e. a measure of the hydrophobicity of a substance. Small values of the log P mean an increased hydrophilicity of the molecule, while large values mean an increased lipophilicity.

It has been found that a molecule with a log P of at least 2.5 has such good lipophilicity or hydrophobicity that the molecule can migrate through the cell wall into cells better than conventional substances with a lower log P, to develop its protection there. Strongly hydrophilic molecules with a log P of less than 2.5, on the other hand, do not pass through the semi-permeable cell wall and so their action is reduced. With a log P value of 2.5 a threshold appears to be reached, which designates substances that are suitable to penetrate into the cells of a tissue in order to prevent ischaemic damage to the tissue by reducing or preventing oxidising factors.

For the adjustment of the log P value, besides the two substituents R₁ and R₂ having a reducing action, the preparation according to the invention also has an additional substituent R₃. This serves to adjust the application properties of the substance in a targeted manner and in particular is varied appropriately in order to adjust the log P value. In detail, the constitution of R₃ is not limited provided that it is biologically compatible and contributes to the hydrophobicity. Thus, R₃ can be either a homoalkyl residue or a heteroalkyl residue, straight-chained or branched. The definition of R₃ comprises substituted and unsubstituted, homoatomic or heteroatomic “residues” in the chemical sense. In a preferred embodiment, the substituent R₃ is an alkyl substituent with a chain length of C6 to C26 and preferably of C8 to C18. In other words, R₃ is preferably a saturated alkyl residue consisting of carbon atoms, which can be linear or branched and comprises 6 to 26 and preferably 8 to 18 carbon atoms. Among the alkyl residues, linear alkyl chains are preferred over branched alkyl chains. It is presumed that these alkyl substituents with a carbon chain of 6 to 26 carbon atoms on that aromatic ring which carries the more hydrophilic substituents R₁ and R₂ significantly increases the hydrophobicity of the aromatic ring so that penetration into the cells is facilitated again, and thus the substance according to the invention can protect these cells and thus the tissue or the organ. The hydrophilic, strongly reducing substituents R₁ and R₂ are therefore “masked”, so to speak, and the substances transferred into the cell, where they develop their action. This action, which increases the lipophilicity, can only occur from a chain length of at least 6 carbon atoms and is most marked with a hydrocarbon residue of 8 to 18 carbon atoms. From a carbon number of more than 26 carbon atoms the substituent R₃ has too marked a screening effect, so that the substance cannot develop its protective action in the cell as the active, strongly reducing functional groups R₁ and R₂ are sterically hindered.

The substituent R₃ can be directly bonded to the aromatic system. In a preferred embodiment, the bonding takes place via a bridging member, which can be the chemical grouping Y—NHCO, where Y represents either a direct bond between the aromatic system and the NHCO group or an alkyl group with a carbon chain of C1 to C8 and preferably of C1 to C3. R₃ is then bonded to the carbonyl carbon. Together with R₃ this therefore represents an amide.

Amides, i.e. substances that have a peptide bond, are frequently found in nature. They are building blocks of the polypeptides, the proteins. It is accordingly presumed that substances having an amide group can in principle migrate into cells very readily.

The inventors have now found that amides which have an alkyl residue with 1 to 8 carbon atoms and preferably from 1 to 3 carbon atoms on the nitrogen atom and an alkyl chain with 2, preferably 6 to 26 carbon atoms on the carbonyl carbon are highly suitable according to the invention to increase the permeability of the substance according to the invention through the cell wall into the interior of the cell, and thus to facilitate entry into the interior of the cell, so that the substance can develop its cell-protecting or organ-protecting action very well in situ. In the case of bonds via a bridging member, the alkyl chain of R₃ can therefore be shorter, since the chain is extended by the bridging member. It is true that longer alkyl chains on the nitrogen atom would also be suitable, but it has been shown that particularly the short alkyl chains, i.e. those comprising a maximum of 3 carbon atoms, are particularly suitable. It is presumed that this is connected with the steric arrangement on the aromatic ring, or also with the electron cloud of the free electron pair on the nitrogen atom, which can bring about a screening effect. The same applies to the alkyl residue which is bonded to the carbonyl carbon. Here, however, the effect of steric screening is no longer as great because the essential screening is already provided by the nitrogen so that, in principle, longer carbon chains of up to 26 C atoms are also possible.

In another preferred embodiment, R₃ is bonded via a group with the structure Y—COO, wherein Y again represents a direct bond between the aromatic system and the COO grouping but can also be a C1 to C8 alkyl group, preferably a C1 to C3 alkyl group. R₃, i.e. preferably an alkyl residue with a chain length of C2 to C26, is bonded to an oxygen atom and in this exemplary embodiment it gives an ester grouping.

Steric approaches similar to those already employed, as already set out in detail for the amides, are applicable to an ester grouping of this type. In addition, esters and amides have a similar polarity, so that they can be used either as alternatives or in combination. However, the peptide bond contributes to a somewhat improved acceptance in the cell or tissue compared with the ester. Moreover, from a chemical viewpoint esters are not as stable as peptides and split into acid and alcohol even at slightly modified pH values, as a result of which the action of the substance according to the invention is at least partly lost.

In another preferred embodiment, R₃ is bonded via a group with the structure Y—CH₂O wherein Y again represents a direct bond between the aromatic system and the CH₂O grouping or can also be a C1 to C8 alkyl group, preferably a C1 to C3 alkyl group. R₃, i.e. preferably an alkyl residue with a chain length of C2 to C26, is bonded to the oxygen, which in this exemplary embodiment leads to an ether grouping.

Ethers with the formula given above may also be considered as a substituent R₃. Through the ether group, the molecule gains a proportion of hydrophilicity, but this is significantly reduced in relation to the esters or amides. Nevertheless, free ethers are found significantly less frequently in the body of a mammal and therefore the acceptance of these substances is somewhat reduced compared with amides and esters. However, this effect is at least partly offset again by the significantly increased lipophilicity so that ethers in the constitution given above also represent an alternative for the substituent R₃.

In another preferred embodiment, at least one of the two substituents R₁ or R₂ carries a protective group. Protective groups for functional groups are always used in chemistry when a particular functional group has to be preserved from premature reaction. After the protective group has been split off, the reactive functional group is free again and can react as desired. The protective groups for OH, SH and NH₂ groups conventionally used in organic chemistry, which are well known to the person skilled in the art, are suitable as protective groups. As is generally known to the person skilled in the art, the protective groups must bond to the functional groups R₁ and R₂ in an adequate manner in order to protect these during storage, but the bond must be formed in such a way that the protective group breaks away again in a physiological environment.

Suitable protective groups for OH are acyl groups, preferably the acetyl group or succinyl group, or phosphate groups. The substance according to the invention is consequently reacted appropriately with a suitable acid, such as e.g. acetic acid or phosphoric acid, when protection of one of the residues R₁ or R₂ is desired. This results in an ester, an amide or a thio ester, depending on the substituent with a reducing action. These protective groups can be split off again very readily, mostly under slightly modified conditions in the interior of the cell, e.g. at a modified pH. As a result of the cleavage of the protective group, the functional group with a strongly reducing action, i.e. either OH, SH or NH₂, is recovered, which protects the cell from oxidative damage in the interior of the cell. Acetyl protective groups are particularly suitable. They are readily obtainable, well known, do not give off any harmful substances when the protective group is removed and are also inexpensive.

Alternatively, it is also possible to use succinyl protective groups or phosphate protective groups. They are obtained by reaction of the substituent(s) R₁ and/or R₂ with succinic acid or phosphoric acid. The succinyl group or phosphate group can also be split off readily again under conditions as present in the interior of a cell, so that the reducing action of the OH, SH or NH₂ groups becomes manifest again. Succinic acid and phosphoric acid, which are recovered after cleavage of the protective group, are also harmless substances to the body which can simply be flushed out again.

Without being bound to the theory, it is assumed that an aromatic system which has at least two substituents R₁ and R₂, selected from OH, SH and NH₂, on a ring has a strongly reducing action and therefore counteracts oxidative damage to cells or tissues, as is brought about by ischaemic conditions.

A very low concentration is sufficient to prevent this damage, i.e. a concentration of the substance of about 0.5 to 200 μM, preferably 1 to 100 μM.

In order to be able to develop the desired protective action, the substance according to the invention can be administered in a variety of ways. All methods of administration are suitable here, such as parenteral or oral administration, with parenteral dosage being preferred. What is essential is for the substance to pass into the blood circulation of the tissue or cells to be protected so that an accumulation of the active substance can take place there in a sufficient quantity. This is usually achieved by injection or infusion into the bloodstream of the donor.

The substance according to the invention is particularly suitable for administration to a donor in the form of an injectable preparation. In this case the preparation consists at least of the substance according to the invention and at least one pharmaceutically acceptable carrier. In the simplest case the carrier can be water. The substance is usually pre-dissolved in suitable pharmaceutically acceptable solvents such as PEG derivatives or similar and then after processing, it is administered either as a solution or dispersion or in the form of liposomes or micelles. It is also possible to use biologically and physiologically compatible surfactants for better processing. The surfactants also used for pharmaceutical products are suitable for this purpose, for example substances marketed with the name “Pluronic”.

The preparation is suitable for injection into a donor and can preferably be used as a flushing solution which flows through the relevant organ to be transplanted so that the substance according to the invention passes into all the cells of the organ. Almost complete irrigation is achieved after about 30 minutes to 2 hours. The preparation preferably contains the substance at a level of 0.5 to 20 μM, as this represents an adequate, effective concentration of the substance according to the invention, i.e. a concentration that protects the cells or the organs.

The substance according to the invention described above is used to protect cells or also tissues and organs. The protection relates in particular to damage by an under-supply of oxygen (ischaemic conditions) to the cells/tissue, particularly in tissues for transplant or removed cells. The substance according to the invention is used in a very low concentration here and displays no haemodynamic activity. It is therefore extremely well tolerated and prolongs the life of cells or tissue intended for transplant sufficiently to reduce or completely prevent damage to the tissue occurring before transplant so that the chances of a successful transplant are significantly increased.

Exemplary embodiments of the invention are described in more detail below.

The active substances are synthesised as described below. The action of the substances on cold-induced damage to cells is quantified in a model system. For this purpose, endothelial cells, e.g. cells of the endothelium of the human umbilical cord vein, are cultured. The cells are incubated with various concentrations of the test substances for variable periods of time and then the medium is replaced by fresh medium without any test substance. Next, the cells are incubated e.g. for 24 hours at 0° C. At the end of the incubation period, the lactate dehydrogenase released is determined in the supernatant of the culture vessels by known methods, this concentration being a measure of cell damage. The effectiveness of the individual compounds is determined by the concentration at which 50% of the release of lactate dehydrogenase is inhibited (EC50).

### Example 1

### N-Octanoyl dopamine

1 gram N-octanoic acid is dissolved in 10 ml tetrahydrofuran and 0.90 grams N-ethyldiisopropylamine are added. While stirring, 0.75 grams (0.658 ml) ethyl chlorocarbonate are added. After 3 hours the mixture is admixed with 15 ml ethyl acetate and 10 ml water. The organic phase is separated off and dried over magnesium sulfate.

Under a nitrogen atmosphere, 1.24 grams dopamine hydrochloride are dissolved in 10 ml dimethylformamide. For this purpose a stoichiometric amount of the ethoxycarbonyl octanoate dissolved in ethyl acetate is added with stirring. The turbidity that forms during this operation disappears again after adding the stoichiometric amount of N-ethyldiisopropylamine. After stirring with the exclusion of light overnight, 20 ml of an aqueous solution with 5% sodium hydrogen carbonate/1% sodium sulfite are added and the organic phase is separated off. The aqueous phase is again extracted with 10 ml ethyl acetate. The combined organic phases are washed consecutively with 10 ml saturated saline solution, 10 ml 0.5 M sulfuric acid and 10 ml saline solution. The organic phase is dried over magnesium sulfate and the solvent is removed in vacuo (rotary evaporator). 1.74 grams (96%) of a very viscous, almost colourless oil are obtained.

### Example 2

### O-Succinyl-N-octanoyl dopamine

0.66 grams N-octanoyl dopamine are dissolved in 3 ml tetrahydrofuran under nitrogen and to this are added 236 mg succinic anhydride in 4 ml tetrahydrofuran. After stirring overnight, the solvent is removed in vacuo and the solid residue is taken up in 5 ml 5% sodium hydrogen carbonate and 5 ml ethyl acetate. The organic phase is discarded and the aqueous phase is admixed with 10 ml ethyl acetate and acidified with 10 ml 0.5 M sulfuric acid. The organic phase is washed with saturated saline solution, dried over sodium sulfate and the solvent is removed in vacuo. Crude O-succinyl-N-octanoyl dopamine is obtainable, which is further purified by recrystallisation.

### Example 3

### N-Decanoyl dopamine

1.72 grams n-decanoic acid are dissolved in 10 ml tetrahydrofuran and 1.2 grams thionyl chloride are added. After adding one drop of dimethylformamide, the mixture is heated to reflux with stirring. After 5 h the solvent is distilled off. 0.95 grams dopamine hydrochloride are dissolved in 6 ml dimethylformamide and the stoichiometric amount of decanoyl chloride is slowly added dropwise in an ice bath under nitrogen. After 3 hours, work-up is performed as in Example 1.

### Example 4

### N-Octadecanoyl dopamine

1.42 grams stearic acid are dissolved in 10 ml tetrahydrofuran and 0.57 g N-hydroxysuccinimide and 1.03 grams dicyclohexylcarbodiimide are added. After stirring overnight, the precipitate is separated off by filtration, washed with tetrahydrofuran and the combined filtrates are freed of solvents in vacuo. Under a nitrogen atmosphere the N-octanoyloxysuccinimide thus obtained is reacted with the stoichiometric amount of dopamine hydrochloride and triethylamine (dissolved in dimethylformamide). After stirring with the exclusion of light overnight, N-octanoyl dopamine is obtained after working up.

### Example 5

### 2,5-Dihydroxybenzoyl amidooctane

From 2.38 grams 2,5-dihydroxybenzoic acid, the acid chloride is prepared in a known manner using phosphorus trichloride. To this, dissolved in 20 ml tetrahydrofuran, the stoichiometric amount of N-octylamine is added slowly under a nitrogen atmosphere in an ice bath while stirring vigorously. On completion of the addition, the ice bath is removed and stirring is continued overnight with the exclusion of light. The solvent is removed in vacuo and the organic phase is washed consecutively with sodium hydrogen carbonate/sodium sulfite solution, water, dilute phosphoric acid and saline solution and finally dried over a molecular sieve. After removal of the solvent, 2,5-dihydroxybenzoyl amidooctane is obtained as a practically white solid.

### Example 6

### 3,4-Dihydroxybenzoyl amidooctane

1.19 grams 3,4-dihydroxybenzoic acid are dissolved in 10 ml tetrahydrofuran and 0.57 grams N-hydroxysuccinimide, 1.03 grams dicyclohexylcarbodiimide and 0.65 grams octylamine are added under nitrogen. After stirring overnight with the exclusion of light the precipitate is filtered off and the organic phase is diluted with 15 ml ethyl acetate and washed with 10 ml 5% sodium hydrogen carbonate/1% sodium sulfite. After shaking with saline solution, 0.5 M sulfuric acid and saline solution, the organic phase was dried over sodium sulfate and freed of the solvent. 1.44 grams (83%) of a beige solid are, obtained.

### Example 7

### 2,5-Bisacetoxybenzoyl amidohexane

From 2,5-dihydroxybenzoic acid, 2,5-bisacetoxybenzoic acid is prepared by known methods with acetic anhydride and sodium acetate. From 1.19 grams of this compound dissolved in 10 ml diethyl ether, the active ester is synthesised by adding 0.68 grams N-hydroxybenzotriazole and 0.96 grams N-(3-dimethylaminopropyl)-N′-ethylcarbodiimide. After stirring overnight the solvent is removed and the residue is taken up in 10 ml ethyl acetate and 10 ml water. The organic phase is dried and 0.5 grams hexylamine are added. After stirring overnight it is washed consecutively with sodium hydrogen carbonate solution, saline solution and dilute phosphoric acid and the organic phase is dried. After removal of the solvent, 1.3 grams (81%) crude 2,5-bisacetoxybenzoyl amidohexane are obtained.

The protective action of some substances according to the invention is represented by their EC50 values (dopamine, adrenalin, noradrenalin and dobutamine only for comparison purposes):

| Substance | EC50 [μM] |
|---|---|
| Dopamine | 75 |
| Adrenalin | 600 |
| Noradrenalin | 700 |
| Dobutamine | 5 |
| N-Octanoyl dopamine | 2.1 ± 0.2 |
| N-Decanoyl dopamine | 0.9 ± 0.2 |
| N-Dodecanoyl dopamine | 1.2 ± 0.2 |
| N-Tetradecanoyl dopamine | 1.3 ± 0.2 |
| N-(4-Methylphenylsulfonyl) dopamine | 12 ± 1 |
| N-(3-Phenylpropanoyl) dopamine | 9 ± 1 |
| 2,3-Dihydroxybenzoyl amidooctane | 1.2 ± 0.1 |
| 3,4-Dihydroxybenzoyl amidooctane | 2.4 ± 0.2 |
| 2,5-Dihydroxybenzoyl amidooctane | 6 ± 1 |

### Example 8

0.5 grams N-octanoyl dopamine are taken up in 9.5 grams of a mixture of 60% (v/v) 1,2-propylene glycol and 40% (v/v) water and mixed. A clear, stable solution is obtained, which is suitable for parenteral application in mammals after sterilisation under the recognised pharmaceutical regulations.

1. A composition for the protection of cells or tissue, comprising an active quantity of a substance dissolved in a physiologically acceptable carrier, wherein the carrier is a carrier based on water or an organic solvent, and optionally a surfactant, characterised in that the substance has an aromatic system having at least one aromatic ring of formula I with two substituents R₁ and R₂, each selected from the group consisting of OH, SH and NH₂, wherein R₁ and R₂ are in ortho or para position to one another, and another substituent R₃ which brings the log P of the molecule to at least 2.5.

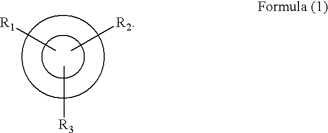

2. The composition according to claim 1, characterised in that the aromatic system has 1 to 3 rings which can be condensed.

3. The composition according to claim 1, characterised in that the aromatic system contains aromatic rings with 5, 6 or 7 carbons.

4. The substance composition according to claim 1, characterised in that R₃ is a substituted or unsubstituted alkyl residue with a chain length of C6 to C26.

5. The composition according to claim 1, characterised in that R₃ is bonded via Y—CH₂O—, Y—COO— or Y—NHC(O)—, wherein Y is a direct bond or a C1 to C8 alkyl group.

6. The composition according to claim 1, characterised in that at least one of the two substituents R₁ or R₂ carries a protective group.

7. The composition according to claim 6, characterised in that the protective group is an acyl group, phosphate group or a succinyl group.

8. (canceled)

9. The composition according to claim 1, characterised in that the carrier is water and that the substance was optionally pre-dissolved in a solubiliser.

10. The composition according to claim 1, characterised in that it the composition contains the substance at a level of 0.5 to 200 μM.

11. The composition according to claim 1, characterised in that the composition is in injectable form.

12. The composition according to claim 1, characterised in that it is present in the form of a dispersion and the substance is contained in the form of micelles or liposomes.

13. (canceled)

14. A method of protecting cells or tissue comprising contacting the cells or tissue in need of protection with an active quantity of a substance having an aromatic system having at least one aromatic ring of formula I with two substituents R₁ and R₂, each selected from the group consisting of OH, SH and NH₂, wherein R₁ and R₂ are in ortho or para position to one another, and another substituent R₃ which brings the log P of the molecule to at least 2.5

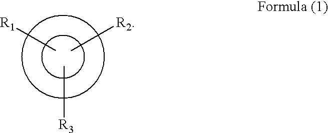

15. The method of claim 14, wherein the contacting is in vivo.

16. The method of claim 14, wherein the contacting is made by injection of the substance.

17. The method of claim 14, wherein the cells or tissue comprises an organ.
